(19) United States
(12) Patent Application Publication
(10) Pub. No.: US 2007/0239101 A1
(43) Pub. Date: Oct. 11, 2007

(54) Method for applying serum to a person's skin

(76) Inventor: David Kellogg, Irvine, CA (US)

Correspondence Address: LAW OFFICES OF ERIC KARICH, 2807 ST. MARK DR., MANSFIELD, TX, 76063, US

(21) Appl. No.: 11/358,572

(22) Filed: Feb. 21, 2006

(51) Int. Cl. A61H 1/00; A61B 17/20

(52) U.S. Cl. 604/22; 601/2

(57) ABSTRACT

A method of treating a person's skin with a serum using an ultrasonic serum application device. The ultrasonic serum application device includes a handle, a paddle that is adapted to removably engage a proximal end of the handle, and an application surface. An ultrasonic vibrator positioned within the handle is operably associated with the application surface for transmitting ultrasonic vibrations to the application surface. The person's skin is coated with the serum and rubbed with the application surface such that the ultrasonic vibrations are transmitted to increase the effectiveness of the serum on the person's skin.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

This invention relates generally to methods for applying a serum to a person's skin, and more particularly to a method for applying a serum using an ultrasonic applicator.

2. Description of Related Art

Cleaning mechanisms that use ultrasonic vibrations to increase their cleaning effectiveness are known in the art, especially in the field of cleaning teeth. An example of such a cleaner is shown in Kleesattell, U.S. Pat. No. 4,281,987, which teaches an ultrasonically driven dental prophylaxis unit.

Ultrasonics are also used for various methods of treating skin. Examples of such uses are shown in the following:

Kellogg, U.S. Pat. No. 6,569,170, teaches a method of cleaning the skin using an ultrasonic cleaning device. Liang et al., U.S. 5,012,797, teaches a method for removing wrinkles using an ultrasonic surgical tool that is adapted to abrade soft tissue.

Bock, WO 97/22325, teaches a method of pre-treating a person's skin using sonic and ultrasonic vibrations to increase the uptake of therapeutic agents through the skin.

Suroff, U.S. Pat. No. 4,040,414, teaches an ultrasonic personal care instrument with a suction device for sucking blockages out of pores to remove blackheads. Other skin cleaning devices have included rotary motor cleaners that are well documented in the art. Examples of this design include Abura et al., U.S. Pat. No. 4,203,431 (facial treatment device), Wolff, U.S. Des. Pat. No. 245,948 (facial cleaning apparatus), Kawada, U.S. Pat. No. 3,906,940 (facial treatment device with oscillating rotary massaging member), Waters et al., U.S. Pat. No. 3,699,952 and Fry et a., U.S. Pat. No. 4,724,563 (skin treating appliance with orbitally driven brush).

Dermabrasions are typically provided using wire brushes, or fine sandpaper. Ultrasonic devices have not been used for providing dermabrasions.

### SUMMARY OF THE INVENTION

The present invention teaches certain benefits in construction and use which give rise to the objectives described below.

The present invention provides a method of treating a person's skin with a serum using an ultrasonic serum application device. The ultrasonic serum application device includes a handle, a paddle that is adapted to removably engage a proximal end of the handle, and an application surface. An ultrasonic vibrator positioned within the handle is operably associated with the application surface for transmitting ultrasonic vibrations to the application surface. The person's skin is coated with the serum and rubbed with the application surface such that the ultrasonic vibrations are transmitted to increase the effectiveness of the serum on the person's skin.

A primary objective of the present invention is to provide a method of treating a person's skin with a serum using an ultrasonic serum application device, the method and device having advantages not taught by the prior art.

Another objective is to provide a method for applying a serum that is simple enough to be done at home with minimal training or expense.

A further objective is to provide an ultrasonic serum application device that utilized ultrasonic vibrations to increase the effectiveness of the serum being applied to the person's skin.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a perspective view of the preferred embodiment of the present invention.

### DETAILED DESCRIPTION OF THE INVENTION

The above-described drawing figures illustrate the invention, a method of treating a person's skin 12 using a ultrasonic serum application device 10 that transmits ultrasonic vibrations through an application surface 30. The ultrasonic serum application device 10 is used to apply a serum 38 onto the person's skin 12.

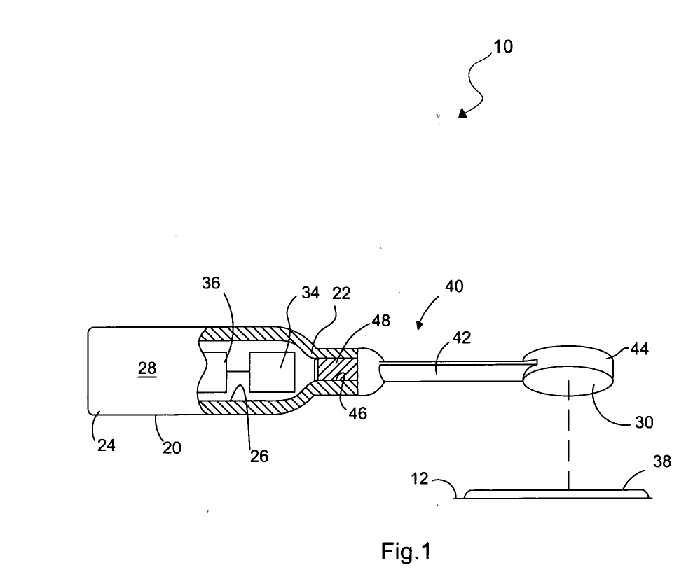

In one embodiment, as shown in FIG. 1, the ultrasonic serum application device 10 includes a handle 20 and a paddle 40 having an application surface 30. The handle 20 has a proximal end 22, a distal end 24, a hollow interior 26, and an exterior gripping surface 28. The handle is preferably formed of molded plastic, although other suitably strong and durable materials may also be used. In alternative embodiments, the handle 20 could also be a softer and more resilient ball shape, or a rigid metal housing, or any other equivalent material or shape that can be devised by those skilled in the art. A rubberized coating (not shown) could also be added to the exterior gripping surface 28 to facilitate gripping the ultrasonic serum application device 10 if it gets wet during use, although this feature is not required.

The paddle 40 is adapted to be mounted at the proximal end 22 of the handle 20, and includes an elongate stem 42 that extends to the application surface 30, which is preferably part of a disk-shaped terminus 44. The elongate stem 42 is preferably relatively rigid and at least slightly resilient, for best transmitting the ultrasonic vibrations.

The handle 20 preferably includes a paddle receiver 46, and the paddle 40 preferably includes an interlocking element 48 that is adapted to removably engage the paddle receiver 46. The interlocking element 48 preferably has a non-circular cross-section to prevent rotation of the paddle 40 with respect to the handle 20.

An ultrasonic vibrator 34 is positioned within the hollow interior 26 and is in operable communication or attachment with the application surface 30 for transmitting ultrasonic vibrations. The ultrasonic vibrator 34 is preferably an eccentrically weighted motor, well known in the prior art, although any means for generating an ultrasonic vibration can be used. The ultrasonic vibrator 34 may be directly attached to the elongate stem 42, or it may be operably attached indirectly through the handle 20 and/or other components, as long as the ultrasonic vibrations are operably transmitted to the application surface 30.

For purposes of this application, the term ultrasonic shall be defined to include any form or vibrations or similar movement that is of sufficient speed and strength to increase the effective uptake of the serum, including ultrasonic vibrations, sonic vibrations, and other forms and speeds of vibration that are operatively effective or equivalent.

The ultrasonic vibrator 34 is operably attached to a means for providing power 36 to the ultrasonic vibrator 34. The means for providing power 36 is preferably a rechargeable battery positioned within the hollow interior 26 and electronically connected to the ultrasonic vibrator 34. In an alternative embodiment, the means for providing power 36 may be a power cord (not shown) adapted to be connected to an external power outlet. Since the means for providing power is not a novel feature of the device, any practical method of powering the ultrasonic vibrator 34 should be considered within the scope of the present invention, and the matter is not described in any greater detail herein.

In use, the person's skin 12 is coated with the serum 38. While we refer specifically to coating the person's skin 12 with the serum 38, this terminology is expressly defined to include adding the serum 38 to the application surface 30 and then using the ultrasonic serum application device 10 to apply the serum 38 to the person's skin 12, or any other similar or equivalent methods.

Ultrasonic vibrations are then initiated in the ultrasonic serum application device 10, the ultrasonic vibrations being transmitted from the ultrasonic vibrator 34, through the application surface 30, and to the person's skin 12 as it is then rubbed with the application surface 30. The ultrasonic vibrations are transmitted to the person's skin 12 such that the serum 38 is effectively transmitted to the person's skin 12.

For purposes of this application, the term serum shall be defined to include any form of skin medication, skin treatment composition, salve, ointment, balm, cream, gel, liniment, rub, or lotion that a person might want to or need to apply to his or her skin.

While the invention has been described with reference to at least one preferred embodiment, it is to be clearly understood by those skilled in the art that the invention is not limited thereto. Rather, the scope of the invention is to be interpreted only in conjunction with the appended claims.

What is claimed is:

1. A method of treating a person's skin with a serum, the method comprising the steps of:
a) providing an ultrasonic serum application device comprising:
a handle having a proximal end, a distal end, a hollow interior, and an exterior gripping surface;
a paddle having an elongate stem that is adapted to removably engage the proximal end of the handle, and a generally planar application surface;
an ultrasonic vibrator positioned within the hollow interior and operably associated with the generally planar application surface for transmitting ultrasonic vibrations from the ultrasonic vibrator to the generally planar application surface; and
a means for providing power to the ultrasonic vibrator;
b) coating the person's skin with the serum;
c) initiating the ultrasonic vibrations of the ultrasonic serum application device, the ultrasonic vibrations being transmitted from the ultrasonic vibrator and through the application surface; and
d) rubbing the person's skin with the application surface such that the ultrasonic vibrations are transmitted to the person's skin to increase the effective penetration of the serum into the person's skin.

2. The method of claim 1 wherein the handle includes a paddle receiver, and wherein the paddle includes an interlocking element that is adapted to removably engage the paddle receiver.

3. The method of claim 2 wherein the interlocking element has a non-circular cross section to prevent rotation of the paddle with respect to the handle.
